(19) United States
(12) Patent Application Publication
(10) Pub. No.: US 2025/0366881 A1
(43) Pub. Date: Dec. 4, 2025

(54) ATHERECTOMY DEVICES, SYSTEMS, AND METHODS

(71) Applicant: BARD PERIPHERAL VASCULAR, INC., Franklin Lakes, NJ (US)

(72) Inventors: Aseem SINGH, Tempe, AZ (US); Austin JONES, Mesa, AZ (US); Alexander HENDRICKSON, Phoenix, AZ (US)

(73) Assignee: BARD PERIPHERAL VASCULAR, INC., Franklin Lakes, NJ (US)

(21) Appl. No.: 18/867,385

(22) PCT Filed: Jun. 16, 2022
(86) PCT No.: PCT/US2022/033773

§ 371 Date: Nov. 19, 2024

(51) Int. Cl. A61B 17/3207; A61B 17/00

(52) U.S. Cl. CPC A61B 17/320725; A61B 17/00234; A61B 2017/00137; A61B 2017/00238; A61B 2017/00305; A61B 2017/00876

(57) ABSTRACT

An atherectomy device including a catheter having a proximal end and a distal end, at least one magnetically actuated bead movably coupled to the catheter at the distal end, the at least one magnetically actuated bead comprising an abrasive surface for performing an atherectomy procedure, a first electromagnet coupled to the distal end of the catheter at a position proximal to the at least one magnetically actuated bead, and a second electromagnet coupled to the distal end of the catheter at a position distal to the at least one magnetically actuated bead. The first electromagnet and the second electromagnet are configured to move the at least one magnetically actuated bead along a length of the catheter.

### TECHNICAL FIELD

The present disclosure relates to atherectomy devices, systems, and methods for removing plaque from a blood vessel, and more particularly atherectomy devices, systems, and methods having magnetically actuated beads that expand a treatment radius and move transversely to remove plaque from a blood vessel.

### BACKGROUND

Atherectomy procedures remove plaque from a body vessel of a patient by abrading the plaque within the body vessel. Traditional orbital or rotational atherectomy devices use rotating abrasive elements that contact calcific plaque in the body vessel while moving, thereby abrading the plaque. The atherectomy devices with rotating abrasive elements may require a user to manually move the abrasive element along the body vessel to abrade the plaque along the body vessel. This may increase the time required to perform the atherectomy procedure. Additionally, traditional orbital atherectomy devices use abrasive beads along a guidewire and increase the rotational velocity of the beads to increase a radius of travel of the abrasive beads to abrade plaque in the body vessel. However, the increased rotational velocity of the abrasive beads can increase the force and contact time on the body vessel, which could potentially result in vessel damage. Accordingly, a need exists for devices and methods for moving the abrasive element along the body vessel without user intervention, and/or increasing the radius of travel of abrasive beads without increasing the velocity of the beads.

### SUMMARY

One challenging aspect of performing an atherectomy procedure is manually moving an abrasive element of an atherectomy device along a body vessel to abrade plaque within the vessel. Embodiments of the present disclosure are directed to systems, methods, and devices for performing atherectomy procedures that include abrasive elements that move along a body vessel without user intervention, as will be described in greater detail below.

In one aspect, for example, an atherectomy device includes a catheter having a proximal end and a distal end, at least one magnetically actuated bead movably coupled to the catheter at the distal end, the at least one magnetically actuated bead comprising an abrasive surface for performing an atherectomy procedure, a first electromagnet coupled to the distal end of the catheter at a position proximal to the at least one magnetically actuated bead, and a second electromagnet coupled to the distal end of the catheter at a position distal to the at least one magnetically actuated bead. The first electromagnet and the second electromagnet are configured to move the at least one magnetically actuated bead along a length of the catheter.

In another aspect, an atherectomy device includes a catheter having a proximal end and a distal end, at least one magnetically actuated bead movably coupled to the catheter at the distal end, the at least one magnetically actuated bead including a shaft, a base having a contact surface, and an abrasive surface for performing an atherectomy procedure, the abrasive surface is coupled to an end of the shaft opposite the base, and an expansion mechanism coupled to the catheter, the expansion mechanism includes a first section having a first thickness, and a second section having a second thickness greater than the first thickness, the expansion mechanism configured to move between a retracted position and an extended position. In the retracted position, the contact surface of the at least one magnetically actuated bead contacts the first section of the expansion mechanism. In the extended position, the contact surface of the at least one magnetically actuated bead contacts the second section of the expansion mechanism. Moving the expansion mechanism from the retracted position toward the extended position moves the at least one magnetically actuated bead away from a centerline of the catheter.

In yet another aspect, there is provided a method of operating an atherectomy device, the method including providing the atherectomy device with a catheter having a proximal end and a distal end; at least one magnetically actuated bead movably coupled to the catheter at the distal end, the at least one magnetically actuated bead comprising an abrasive surface for performing an atherectomy procedure; a first electromagnet coupled to the distal end of the catheter at a position proximal the at least one magnetically actuated bead; and a second electromagnet spaced coupled to the distal end of the catheter at a position distal to the at least one magnetically actuated bead, the first electromagnet and the second electromagnet are configured to move the at least one magnetically actuated bead along a length of the catheter; and supplying an electrical current to the first electromagnet and the second electromagnet to move the at least one magnetically actuated bead along the length of the catheter.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a perspective view of an atherectomy device, according to one or more embodiments shown and described herein;

FIG. 2 schematically depicts a cross-sectional side view of the atherectomy device of FIG. 1 taken along lines 2-2 in an extended position, according to one or more embodiments shown and described herein;

FIG. 3A schematically depicts the atherectomy device of FIG. 2 in a retracted position, according to one or more embodiments shown and described herein;

FIG. 3B schematically depicts an axial cross-sectional view taken along lines 3B-3B of the atherectomy device of FIG. 1 in a retracted position, according to one or more embodiments shown and described herein;

FIG. 4A schematically depicts the cross-sectional side view of the atherectomy device of FIG. 2 in a partially extended position, according to one or more embodiments shown and described herein;

FIG. 4B schematically depicts the axial cross-sectional view of the atherectomy device of FIG. 3B in the partially extended position, according to one or more embodiments shown and described herein;

FIG. 5 schematically depicts the axial cross-sectional view of the atherectomy device of FIG. 4B in the extended position, according to one or more embodiments shown and described herein;

FIG. 6 schematically depicts a controller for operating the atherectomy device of FIG. 1, according to one or more embodiments shown and 108 described herein;

FIG. 7 depicts a flowchart illustrating a method of operating the atherectomy device of FIG. 1, according to one or more embodiments shown and described herein; and

FIG. 8 schematically depicts an axial cross-sectional front view of another embodiment of an atherectomy device, according to one or more embodiments shown and described herein.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Embodiments described herein are directed to devices, systems, and methods for use in performing an atherectomy procedure. For example, in some embodiments, an atherectomy device includes a catheter and at least one magnetically actuated bead including an abrasive surface. The magnetically actuated bead may be driven longitudinally between first and second electromagnets to allow the magnetically actuated bead to perform an atherectomy operation. Accordingly, movement of the magnetically actuated bead may be controlled via operation of the electromagnets as opposed to manual back and forth actuation, thereby improving atherectomy procedures. For example, an atherectomy procedure may require less effort and control via a user, allowing more predictable and consistent results. These and additional features and benefits will be described in greater detail herein.

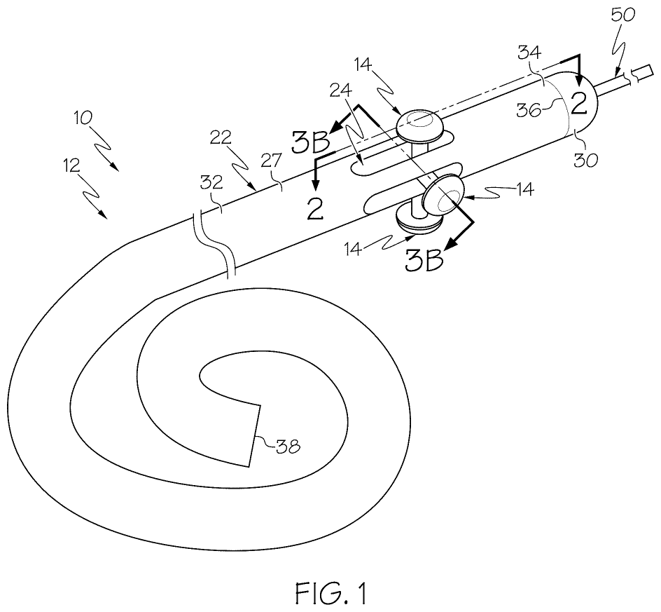

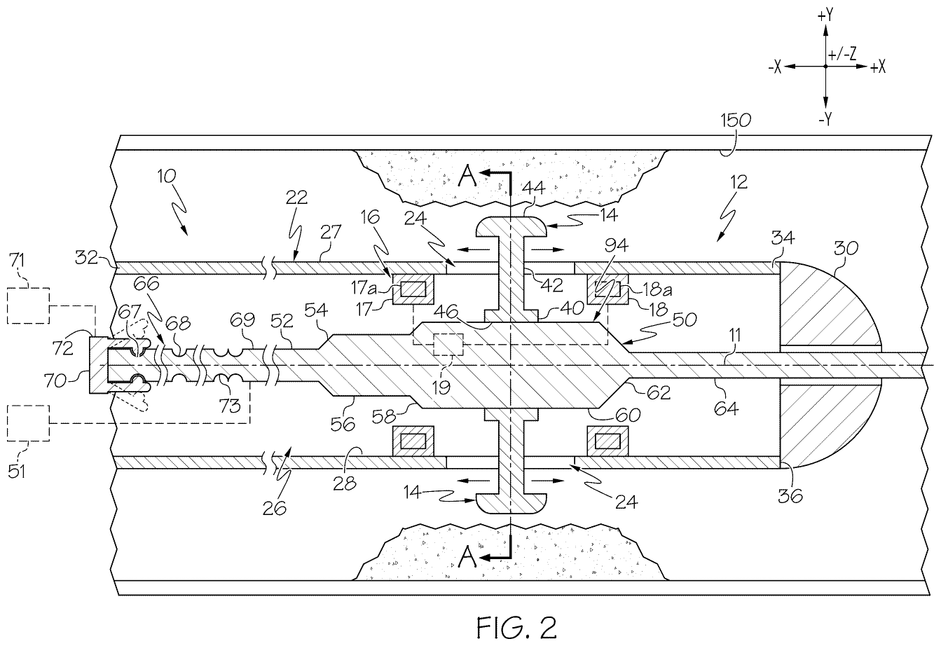

Referring to FIG. 1, an atherectomy device 10 for performing an atherectomy procedure is schematically depicted. The atherectomy device 10 may be inserted into a body vessel 150 of a patient, as schematically depicted in FIG. 2. The body vessel 150 may be, for example, a blood vessel. The atherectomy device 10 is configured to remove plaque from the body vessel 150 by abrading the plaque to thereby increase blood flow through the body vessel 150 that was previously restricted by the plaque buildup.

The atherectomy device 10 may generally include a catheter 12, one or more magnetically actuated beads 14, a magnetic drive mechanism 16, an expansion mechanism 50, and a locking mechanism 70. The catheter 12 may include a catheter body 22 with a sidewall 27, a lumen 26 defined by an interior surface 28 of the catheter body 22, a plurality of openings 24 extending through the sidewall 27 of the catheter body 22 to the lumen 26, a proximal portion 32 positioned proximal to the openings 24, a distal portion 34 positioned distal to the openings 24, a distal tip 30 coupled to the distal portion 34 at a distal end 36 of the catheter body 22, and a proximal end 38 opposite the distal end 36. The lumen 26 may extend along a length of the catheter body 22, such that the catheter body 22 is hollow. The proximal end 38 of the catheter 12 may be operatively coupled to a controller (not shown) capable of operating the atherectomy device 10. The openings 24 may be positioned at or proximal the distal end 36 of the catheter 12. The catheter body 22 may be substantially cylindrical. The catheter body 22 may be formed of a flexible material, such as plastic, rubber, or the like, to allow the catheter body 22 to bend when moving through tortuous vessels. The openings 24 may be elongated and extend along an axial direction (e.g., in the +/−X direction) of the catheter 12, such that the openings 24 act as a set of tracks for the magnetically actuated beads 14 to move along. The openings 24 may be narrower in a radial direction (e.g., in the +/−Z direction) than in the axial direction to restrict movement of the magnetically actuated beads 14 substantially in the axial direction and the radial direction (e.g., in the +/−Z direction and/or in the +/−Y direction) only.

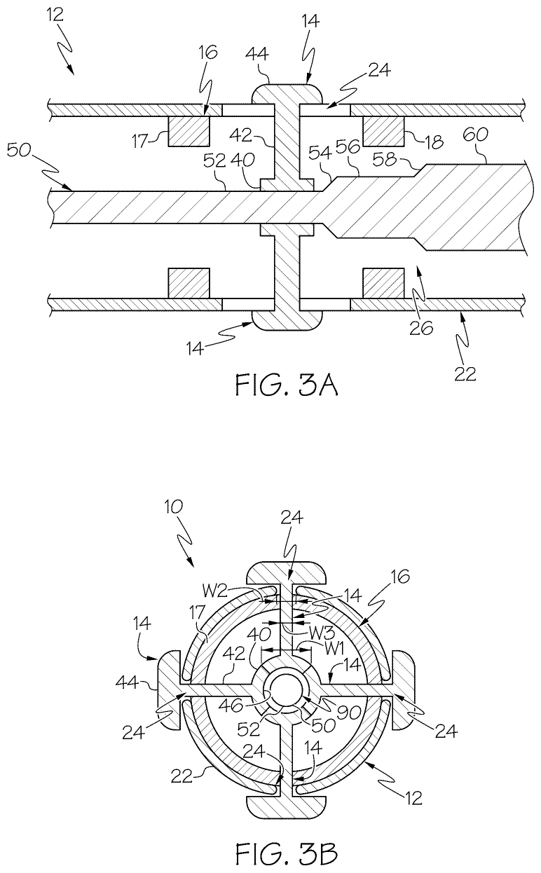

The magnetically actuated beads 14 may each include a base 40, a shaft 42 extending in a radial direction (e.g., in the +/−Z direction and/or in the +/−Y direction) from the base 40, and an abrasive surface 44 for performing an atherectomy procedure. The abrasive surface 44 may be any shape and/or formed of any material capable of performing an atherectomy procedure, specifically, abrading plaque in a body vessel. For example, the abrasive surface 44 may include a diamond coating, or be shaped as a bumpy or spiky surface. The abrasive surface 44 may be coupled to an end of the shaft 42 opposite the base 40. As shown in FIG. 3B, the base 40 may have a width W1 that is greater than a width W2 of the openings 24. The shaft 42 may have a width W3 that is less than the width W2 of the openings 24 to allow the shaft 42 to extend through the openings 24 and allowing axial movement of the magnetically actuated beads 14 relative to the catheter body 22. The base 40 may be positioned within the lumen 26 of the catheter 12. The abrasive surface 44 may be positioned outside of the lumen 26 of the catheter 12. The base 40 may include a contact surface 46 configured to contact the expansion mechanism 50. The base 40 and the contact surface 46 may be shaped to complement the shape of the expansion mechanism 50. The contact surface 46 may include a shape to complement the shape of the expansion mechanism 50, such as round, flat, multi-planar, or the like. In other embodiments, the contact surface 46 may have a shape which does not complement the shape of the expansion mechanism 50.

The magnetically actuated beads 14 may be biased toward the expansion mechanism 50 to maintain contact between the contact surface 46 of the magnetically actuated beads 14 and the expansion mechanism 50. The magnetically actuated beads 14 may be biased toward the expansion mechanism 50 via pressure exerted on the abrasive surfaces 44 of the magnetically actuated beads 14 from walls of the body vessel 150. Alternatively or additionally, the atherectomy device 10 may include a biasing mechanism that biases the magnetically actuated beads 14 toward the expansion mechanism 50 to maintain contact between the contact surface 46 of the base 40 and the expansion mechanism 50. In some embodiments, the biasing mechanism may be a set of springs coupled between the magnetically actuated beads 14 and the expansion mechanism 50.

The atherectomy device 10 may include two pairs of magnetically actuated beads 14 that may each be positioned radially diametrically opposite one another across the centerline 11 of the catheter 12, such that the magnetically actuated beads 14 are equidistantly disposed about the circumference of the catheter 12. The magnetically actuated beads 14 may be formed at least partially of a magnetic material that allows the magnetically actuated beads 14 to be moved via magnetic forces created by the magnetic drive mechanism 16. For example, the magnetically actuated beads 14 may be formed of a magnetic material, or a metal capable of being attracted or repelled by magnets, such as a ferromagnetic metal.

The magnetic drive mechanism 16 may include a first electromagnetic junction 17, a second electromagnetic junction 18, and may be connectable or connected to a current source 19. In embodiments, the first electromagnetic junction 17 and the second electromagnetic junction 18 may each include individual electromagnets 17a, 18a positioned about the lumen 26. As used herein, an “electromagnet” refers to a type of magnet in which a magnetic field is produced by an electric current. For example, each junction 17 and 18 may include a conductive wire that is wound into a coil. A current through the wire creates a magnetic field having a north and south poles. The electromagnetic junctions 17, 18 may be a location or an assembly of one or more electromagnets that attract/repel the magnetically actuated beads 14 toward the location of the electromagnets. For example, the magnetically actuated beads 14 may have opposite N and S poles that are located nearer a respective one of the first and second electromagnetic junctions 17 and 18. In embodiments, the first electromagnetic junction 17 and the second electromagnetic junction 18 may each include one or more electromagnets 17a, 18a for each of the openings 24 encapsulated by or housed within material forming the first and second electromagnetic junctions 17 and 18. The material forming the first and second electromagnetic junctions 17 and 18 may enhance the magnetic field provided by the electromagnets by increasing a magnetic force between the electromagnetic and magnetic components. For example, the first electromagnetic junction 17 may include four electromagnets when the catheter body 22 includes four openings 24, and the electromagnets may be positioned adjacent to one of the openings 24 in a 1:1 ratio. In embodiments, the magnetic drive mechanism 16 may include more than two electromagnets. In some embodiments, the electromagnets 17a, 18a themselves may form the junctions 17 and 18. In embodiments, a single electromagnet pair may be included to drive the plurality of magnetically actuated beads 14.

In some embodiments, the magnetic drive mechanism 16 may include only a single electromagnetic junction. In these embodiments, a biasing member, such as a spring, may be used to push or pull the magnetically actuated beads 14 in the longitudinal direction (e.g., in the +/−X direction) back to an initial position after the electromagnet has been actuated and then deactivated. The attraction/repulsion and push/pull of the biasing member can be done repeatedly to achieve the back and forth motion of the magnetically actuated beads 14. In other words, a single electromagnetic junction may be provided distally or proximally to the magnetically actuated beads 14, which is actuatable to attract or repel the magnetically actuated beads 14, and a biasing member such as a spring may be provided to provide an opposing force to the electromagnetic junction.

The first electromagnetic junction 17 and the second electromagnetic junction 18 may be coupled to the catheter body 22 of the catheter 12. The first electromagnetic junction 17 may be positioned at and coupled to the proximal portion 32 of the catheter body 22 such that the first electromagnetic junction 17 may be positioned proximal to the magnetically actuated beads 14. The second electromagnetic junction 18 may be positioned at and coupled to the distal portion 34 of the catheter body 22 such that the second electromagnetic junction 18 may be positioned distal to the magnetically actuated beads 14. The openings 24 may be positioned between the first electromagnetic junction 17 and the second electromagnetic junction 18.

The first electromagnetic junction 17 and the second electromagnetic junction 18 may include the electromagnets 17a, 18a that are positioned within the lumen 26 of the catheter body 22 as shown in FIG. 1, or alternatively, may be positioned outside of the catheter body 22 or embedded in the side wall of catheter body 22. Each of the first electromagnetic junction 17 and the second electromagnetic junction 18 may be disk-shaped, such that the expansion mechanism 50 may extend therethrough. However, it is contemplated and possible that the first electromagnetic junction 17 and the second electromagnetic junction 18 may have any shape capable of allowing the expansion mechanism 50 to extend therethrough or along the first electromagnetic junction 17 and the second electromagnetic junction 18. The first electromagnetic junction 17 and the second electromagnetic junction 18 may be spaced apart on opposing sides of the openings 24 of the catheter body 22. The first electromagnetic junction 17 may be positioned at the proximal portion 32 of the catheter 12 proximal to the openings 24, and the second electromagnetic junction 18 may be positioned at the distal portion 34 of the catheter 12 distal to the openings 24. Each of the first electromagnetic junction 17 and the second electromagnetic junction 18 may include the electromagnets 17a, 18a that are electrically coupled to the current source 19 such that the current source 19 may supply a current to each of the first electromagnetic junction 17 and the second electromagnetic junction 18.

The current source 19 may supply a current individually to the electromagnets 17a, 18a of each of the first electromagnetic junction 17 and the second electromagnetic junction 18. The current source 19 may provide a first current to the electromagnets 17a of the first electromagnetic junction 17 and may supply a second current to electromagnets 18a of the second electromagnetic junction 18. The first electromagnetic junction 17 and the second electromagnetic junction 18 are configured to move the magnetically actuated beads 14 along a length of the catheter 12 when a current is applied to either of the electromagnets 17a, 18a of the first electromagnetic junction 17 and the second electromagnetic junction 18. When the first current is applied to the electromagnets 17a of the first electromagnetic junction 17, the first electromagnetic junction 17 may attract or repel the magnetically actuated beads 14 toward or away from the first electromagnetic junction 17, respectively, due to the natural polarity of the magnetically actuated beads 14 and the polarity of the electromagnets 17a. When the second current is applied to the electromagnets 18a of the second electromagnetic junction 18, the second electromagnetic junction 18 may attract or repel the magnetically actuated beads 14 toward or away from the second electromagnetic junction 18, respectively. The current source 19 may alternate delivery of the current between the first electromagnetic junction 17 and the second electromagnetic junction 18 to oscillate the magnetically actuated beads 14 between the first electromagnetic junction 17 and the second electromagnetic junction 18. For example, the current source 19 may supply the first current to the first electromagnetic junction 17 without supplying the second current to the second electromagnetic junction 18 to move the magnetically actuated beads 14 toward the first electromagnetic junction 17. When the magnetically actuated beads 14 are positioned nearer to the first electromagnetic junction 17 than the second electromagnetic junction 18, the current source 19 may supply the second current to the second electromagnetic junction 18 without supplying the first current to the first electromagnetic junction 17, thereby moving the magnetically actuated beads 14 toward the second electromagnetic junction 18. The current source 19 may alternate between supplying the first current and the second current to oscillate the magnetically actuated beads 14 between the electromagnets. In some embodiments, the current source 19 may switch the polarity of the electromagnets 17a, 18a by reversing the current direction. The current source 19 may provide either of an alternating current or a direct current.

The current source 19 may increase or decrease the current flowing to the electromagnetic junctions 17, 18 to increase or decrease the magnetic force from the first electromagnetic junction 17 and/or second electromagnetic junction 18. Specifically, the current source 19 may increase the current to increase the magnetic force from the electromagnets on the magnetically actuated beads 14. Similarly, the current source 19 may decrease the current to decrease the magnetic force from the electromagnets on the magnetically actuated beads 14. In embodiments, the magnetic drive mechanism 16 may include any number of electromagnets, such as one, two, three, four, and the like. In embodiments, the current sent from the current source 19 to the electromagnets of the electromagnetic junctions 17, 18 may be varied, for example selectively reversed, to individually attract and repel the magnetically actuated beads 14 to the first electromagnetic junction 17 and/or the second electromagnetic junction 18 magnetically actuated beads 14. In such embodiments, the magnetic drive mechanism 16 may include a single electromagnet positioned either proximal or distal to the openings 24 to which the current source 19 is configured to supply current in both directions, such that the single electromagnet may selectively attract and repel the magnetically actuated beads 14.

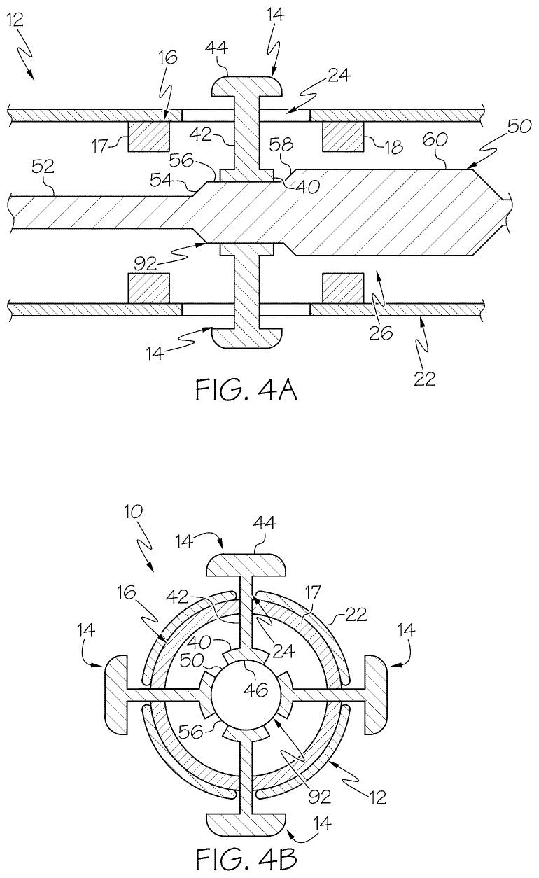

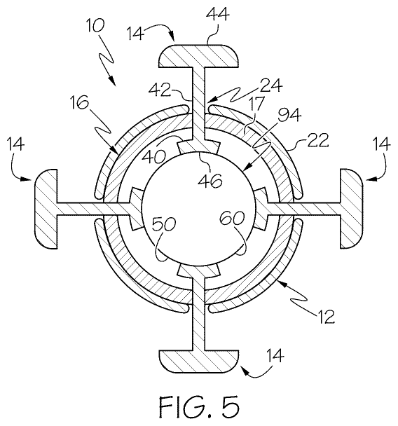

The expansion mechanism 50 may be positioned within the lumen 26 of the catheter 12, and sized so as to be movable through the catheter 12. The expansion mechanism 50 may be configured to move in the longitudinal direction (e.g., in the +/−X direction) along a length of the catheter 12 between a retracted position 90 (FIGS. 3A and 3B), a partially extended position 92 (FIGS. 4A and 4B), and an extended position 94 (FIGS. 2 and 5). When the expansion mechanism 50 is moved to the retracted position 90, the magnetically actuated beads 14 are radially displaced from the centerline 11 of the catheter 12. The expansion mechanism 50 may be operatively coupled to an actuator 51 that moves the expansion mechanism 50 through the catheter 12. The actuator 51 may be a linear actuator, a rotary actuator, a hydraulic actuator, a manual actuator, or the like. Operation of the actuator 51 may move the expansion mechanism 50 between the retracted position 90, the partially extended position 92, and the extended position 94. In embodiments, the expansion mechanism 50 may extend out of the proximal end 38 of the catheter 12, such that the expansion mechanism 50 may be physically manipulated by a user's hand to move the expansion mechanism 50 between the retracted position 90, the partially extended position 92, and the extended position 94. In embodiments, the expansion mechanism 50 may extend out of the distal tip 30 of the catheter 12 to operate as a guidewire. In other embodiments, the expansion mechanism 50 may define a lumen extending therethrough configured to receive a guidewire that may extend through the expansion mechanism 50 and out of the proximal end 38 of the catheter 12.

The expansion mechanism 50 may be a tiered structure including a plurality of sections with different thicknesses. The expansion mechanism 50 may include a first section 52, a second section 56, a third section 60, tapered portions 54, 58, 62 positioned between each section, a distal extension 64, and a locking feature 66. The tapered portions 54, 58, 62 may have a varying thickness, such that the thickness of the expansion mechanism 50 is tapered in the tapered portions 54, 58, 62 between each section. For example, the tapered portion 54 between the first section 52 and the second section 56 may have a variable, increasing thickness as the tapered portion 54 extends from the first section 52 to the second section 56. In other words, a surface of the tapered portions 54, 58, 62 may extend in the axial direction and the radial direction.

The locking feature 66 may be formed in the first section 52 of the expansion mechanism 50. The first section 52, the second section 56, and the third section 60 may extend substantially parallel to the longitudinal direction. The first section 52 may include a first thickness. The second section 56 may include a second thickness. The third section 60 may include a third thickness. The second thickness of the second section 56 may be greater than the first thickness of the first section 52. The third thickness of the third section 60 may be greater than the second thickness of the second section 56. The distal extension 64 may include a distal thickness that is less than the third thickness of the third section 60. The distal thickness may be substantially equal to the first thickness of the first section 52.

Referring to FIGS. 3A-5, the expansion mechanism 50 is depicted in each of the retracted position 90, the partially extended position 92, and the extended position 94. The expansion mechanism 50 may have a rounded cross-section when viewed along the axial direction. Each of the first section 52, the second section 56, the third section 60, and distal extension 64 may be cylindrical in shape. However, in embodiments, each of the sections may have a cross-section of any suitable shape capable of interfacing with the contact surface 46 of the base 40 of the magnetically actuated beads 14, such as triangular, rectangular, pentagonal, and the like. The cross-sectional shape may correspond to the number of magnetically actuated beads 14. For example, in embodiments including three magnetically actuated beads 14, the cross-sectional shape of the sections 52, 56, 60 may be triangular. Each of the tapered portions 54, 58, 62 may be shaped as a truncated cone. However, in embodiments, each of the tapered portions 54, 58, 62 may have a cross-sectional shape corresponding to the cross-sectional shape of the sections.

The expansion mechanism 50 may be movable along the length of the catheter 12 between a retracted position 90, a partially extended position 92, and an extended position 94. Referring to FIG. 3A, in the retracted position 90, the first section 52 may be at least partially disposed between the electromagnets. The openings 24 may be entirely disposed radially outward of the first section 52. In other words, an orthographic projection of the openings 24 extending in the radial direction may entirely intersect the first section 52.

Referring to FIGS. 4A and 4B, in the partially extended position 92, the second section 56 may be at least partially disposed between the electromagnets. The openings 24 may be entirely disposed radially outward of the second section 56. In other words, an orthographic projection of the openings 24 extending in the radial direction may entirely intersect the second section 56. When moving from the retracted position 90 to the partially extended position 92, the magnetically actuated beads 14 may be moved radially outward via contact between the contact surface 46 of the beads 14 and the tapered portion 54 between the first section 52 and the second section 56. In the partially extended position 92, the abrasive surface 44 of the magnetically actuated beads 14 may be disposed further radially outward than when in the retracted position 90.

Referring to FIG. 5, in the extended position 94, the third section 60 may be at least partially disposed between the electromagnets. The openings 24 may be entirely disposed radially outward of the third section 60. In other words, an orthographic projection of the openings 24 extending in the radial direction may entirely intersect the third section 60. When moving from the partially extended position 92 to the extended position 94, the magnetically actuated beads 14 may be moved radially outward via contact between the contact surface 46 of the beads 14 and the tapered portion 58 between the second section 56 and the third section 60. In the extended position 94, the abrasive surface 44 of the magnetically actuated beads 14 may be disposed further radially outward than when in the retracted position 90 and the partially extended position 92.

Referring again to FIG. 2, the locking feature 66 may be positioned on a proximal extension 69 of the expansion mechanism 50 opposite the distal extension 64. In embodiments, the locking feature 66 may be positioned along the expansion mechanism 50, such as, for example, on the distal extension 64, the proximal extension 69, or the like. The first section 52, the second section 56, and the third section 60 may be positioned between the distal extension 64 and the proximal extension 69 of the expansion mechanism 50. The locking feature 66 may be a detent formed within the expansion mechanism 50. The detent may be shaped to correspond to an engaging member 72 of the locking mechanism 70, as will be described in further detail below. The detent may be an indentation formed in the expansion mechanism 50. The detent may be a pair of indentations formed in the expansion mechanism 50, the indentations being positioned adjacent one another. The locking feature 66 may include a set of detents, such as three detents. The set of detents may include a first detent 67, a second detent 68 spaced apart from the first detent 67, and a third detent 73 spaced apart from the second detent 68 and the first detent 67. The second detent 68 may be positioned closer to the first section 52 than the first detent 67 and the third detent 73 may be positioned closer to the first section 52 than the second detent 68. The first detent 67, the second detent 68, and the third detent 73 may be positioned along the expansion mechanism 50 to correspond to one of the positions of the expansion mechanism 50. In other words, the first detent 67 may be positioned along a length of the expansion mechanism 50 such that the expansion mechanism 50 is in the retracted position 90 when the engaging member 72 of the locking mechanism 70 engages the first detent 67. Similarly, the second detent 68 may be positioned along a length of the expansion mechanism 50 such that the expansion mechanism 50 is in the partially extended position 92 when the engaging member 72 of the locking mechanism 70 engages the second detent 68, and the third detent 73 may be positioned along a length of the expansion mechanism 50 such that the expansion mechanism 50 is in the extended position 94 when the engaging member 72 of the locking mechanism 70 engages the third detent 73.

The first detent 67 and the second detent 68 may each be a single indentation. The third detent 73 may be a pair of indentations. However, in embodiments, each of the first detent 67, the second detent 68, and the third detent 73 may include any operable number of indentations for the engaging member 72 of the locking mechanism 70 to engage. The third detent 73 may include a number of detents or a shape different from the first detent 67 and the second detent 68 to provide an indication that the engaging member 72 of the locking mechanism 70 has engaged the third detent 73. The locking feature 66 may include any number of detents. In embodiments, the locking feature 66 includes a single detent. The detents may be formed at least partially along the circumference of the expansion mechanism 50. The detents may be formed entirely along the circumference of the expansion mechanism 50.

Referring still to FIG. 2, the locking mechanism 70 may be configured to restrict movement of the expansion mechanism 50. The locking mechanism 70 may be moveable between a locked position and an unlocked position (shown in phantom). For example, the locking mechanism 70 may include hinged or flexible arms that may move between the two positions to engage the expansion mechanism 50 to restrict movement of the expansion mechanism 50. In the locked position, the engaging member 72 of the locking mechanism 70 may extend into one of the first detent 67, the second detent 68, and the third detent 73 of the locking feature 66 to engage the expansion mechanism 50, thereby restricting movement of the expansion mechanism 50 relative to the locking mechanism 70. The locking mechanism 70 may be fixedly coupled to the catheter 12 to fix the position of the locking mechanism 70 relative to the catheter 12. The engaging member 72 may pivot between the locked position and the unlocked position. In embodiments, the engaging member 72 may be a clamp that clamps onto the expansion mechanism 50 to engage the expansion mechanism 50. In such embodiments, the locking feature 66 may not include detents. Further, in such embodiments, the locking feature 66 may be a flat surface on the expansion mechanism 50, where the engaging member 72 clamps onto the flat surface. In the unlocked position, the locking mechanism 70 may disengage the first detent 67, the second detent 68, and the third detent 73 of the locking feature 66 of the expansion mechanism 50, where the engaging member 72 is spaced apart from the expansion mechanism 50, thereby permitting movement of the expansion mechanism 50.

The locking mechanism 70 may be operatively coupled to a locking actuator 71 that moves the locking mechanism 70. Particularly, for example, the locking actuator 71 may be fixedly coupled between the catheter 12 and the engaging member 72 to be configured to move the engaging member 72 relative to the catheter 12 between the locked position and the unlocked position. For further example, the locking mechanism 70 and locking actuator 71 may be an electromagnet connected to a current source which moves the engaging member 72 between the locked position and the unlocked position. The locking actuator 71 may be a linear actuator, a rotary actuator, a hydraulic actuator, a manual actuator, or the like. Operation of the locking actuator 71 may move the locking mechanism 70 between the locked position and the unlocked position. Operation of the locking actuator 71 may move the engaging member 72 into engagement with the locking feature 66.

The locking mechanism 70 may be actuated before operation of the magnetic drive mechanism 16 to maintain the expansion mechanism 50 in a single position during operation of the magnetic drive mechanism 16 and movement of the magnetically actuated beads 14. By maintaining the expansion mechanism 50 in a single position, the magnetically actuated beads 14 contact a single section of the expansion mechanism 50 during operation.

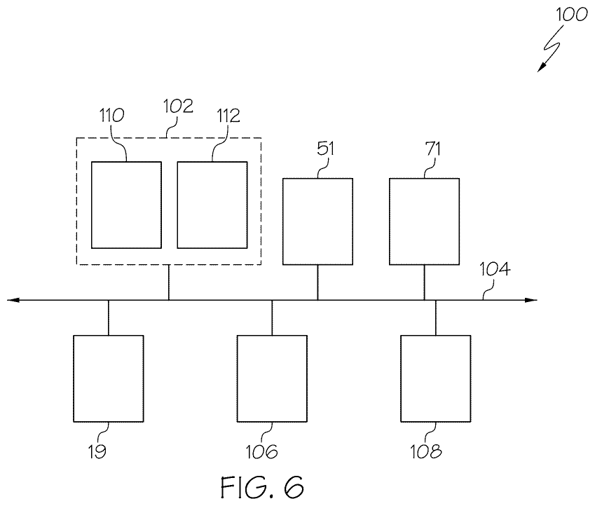

Referring to FIG. 6, the atherectomy device 10 may further include a control system 100. The control system 100 may be positioned within a handle coupled to the proximal end of the catheter 12. The control system 100 may include an electronic control unit (ECU) 102, a communication path 104, a sensor 106, and an input 108. The ECU 102 may be communicatively coupled to the current source 19, the actuator 51, the locking actuator 71, the sensor 106, and the input 108 via the communication path 104 such that the ECU may control operation of the magnetic drive mechanism 16, the position of the expansion mechanism 50, the position of the locking mechanism 70, and receive signals from the sensor 106 and the input 108. The ECU 102 may be configured to control the amount of current from the current source 19 to selectively provide the current from the current source 19 to the first electromagnetic junction 17 and the second electromagnetic junction 18. By controlling the amount of current, the ECU 102 may control a speed, or oscillating frequency, of the magnetically actuated beads 14 at which the magnetically actuated beads 14 move along the length of the catheter 12.

The communication path 104 may provide data interconnectivity between various modules disposed within the atherectomy device 10. Such modules may include, for example, the current source 19, the ECU 102, the sensor 106, and the input 108. Specifically, each of the modules can operate as a node that may send and/or receive data and may each include a processor, such as a microprocessor, configured to send and/or receive data regarding the operation of the module. In some embodiments, the communication path 104 may include a conductive material that permits the transmission of electrical data signals to and between processors, memories, sensors, and valves, pumps, etc. throughout the atherectomy device 10. In another embodiment, the communication path 104 can be a bus, such as for example a LIN bus, a CAN bus, a VAN bus, and the like. In further embodiments, the communication path 104 may be wireless and/or an optical waveguide. Components that are communicatively coupled may include components capable of exchanging data signals with one another such as, for example, electrical signals via conductive medium, electromagnetic signals via air, optical signals via optical waveguides, and the like.

Still referring to FIG. 6, the ECU 102 may be configured to selectively operate components of the atherectomy device 10. For example, the ECU 102 may control the current source 19 to send a current to the first electromagnetic junction 17 and the second electromagnetic junction 18 to move the magnetically actuated beads 14 between the first electromagnetic junction 17 and the second electromagnetic junction 18. The ECU 102 may control the actuator 51 to move the expansion mechanism 50 between the retracted position 90, the partially extended position 92, and the extended position 94. Similarly, the ECU 102 may control the locking actuator 71 to move the locking mechanism 70 between the locked position and the unlocked position. For example, the ECU 102 may be configured to control the operation of a motor for operating the actuator 51 and a motor for operating the locking actuator 71.

The ECU 102 may include one or more processors 110 and one or more memory modules 112. The one or more processors 110 may include any device capable of executing computer-readable executable instructions stored on a non-transitory computer-readable medium. Accordingly, each processor may include a controller, an integrated circuit, a microchip, a computer, and/or any other computing device. It is noted that the one or more processors 110 may reside within the atherectomy device 10 and/or external to the atherectomy device 10.

The one or more memory modules 112 are communicatively coupled to the one or more processors 110 over the communication path 104. The one or more memory modules 112 may be configured as volatile and/or nonvolatile memory and, as such, may include random access memory (including SRAM, DRAM, and/or other types of RAM), flash memory, secure digital (SD) memory, registers, compact discs (CD), digital versatile discs (DVD), and/or other types of non-transitory computer-readable mediums. Depending on the particular embodiment, these non-transitory computer-readable mediums may reside within the atherectomy device 10 and/or external to the atherectomy device 10. The one or more memory modules 112 may be configured to store one or more pieces of logic to selectively operate the current source 19. In some embodiments, the one or more memory modules 112 may be configured to store one or more pieces of logic to selectively operate the atherectomy device 10.

Embodiments of the present disclosure include logic stored on the one or more memory modules 112 that includes machine-readable instructions and/or an algorithm written in any programming language of any generation (e.g., 1 GL, 2 GL, 3 GL, 4 GL, and/or 5 GL) such as, machine language that may be directly executed by the one or more processors 110, assembly language, obstacle-oriented programming (OOP), scripting languages, microcode, etc., that may be compiled or assembled into machine readable instructions and stored on a machine readable medium. Similarly, the logic and/or algorithm may be written in a hardware description language (HDL), such as logic implemented via either a field-programmable gate array (FPGA) configuration or an application-specific integrated circuit (ASIC), and their equivalents.

Accordingly, the logic may be implemented in any conventional computer programming language, as pre-programmed hardware elements, and/or as a combination of hardware and software components. The processor 110 may execute the computer-readable executable instructions, causing the ECU 102 to automatically cause the current source 19 to, for example, alternate delivery of current between the first electromagnetic junction 17 and the second electromagnetic junction 18 to oscillate the magnetically actuated beads 14 between the first electromagnetic junction 17 and the second electromagnetic junction 18. The ECU 102 may further be configured to control an amount of the current from the current source 19 to selectively provide the current from the current source 19 to the first electromagnetic junction 17 and the second electromagnetic junction 18. Controlling the amount of the current from the current source 19 may control a speed of the magnetically actuated beads 14. Specifically, increasing the amount of the current may increase the speed of the magnetically actuated beads 14, and decreasing the amount of the current may decrease the speed of the magnetically actuated beads 14. Similarly, the processor 110 may execute the computer-readable executable instructions, causing the ECU 102 to automatically cause the expansion mechanism 50 to move between the retracted position 90, the partially extended position 92, and the extended position 94.

Still referring to FIG. 6, the sensor 106 may be configured to detect whether the locking mechanism 70 is engaged with the locking feature 66 of the expansion mechanism 50. Specifically, the sensor 106 may be configured to detect whether the expansion mechanism 50 is locked in place. The sensor 106 may be any sensor for detecting the position of the locking mechanism 70, such as for example, a hall effect sensor, a camera, a capacitive inductance sensor, or the like. The ECU 102 may receive a signal from the sensor 106 indicative of whether the locking mechanism 70 is in the locked position. The ECU 102 may be configured to prevent operation of the atherectomy device 10 when the ECU 102 determines that the locking mechanism 70 is not in the locked position. The ECU 102 may prevent operation of the atherectomy device 10 by preventing the current source 19 from sending a current to either of the first electromagnetic junction 17 and the second electromagnetic junction 18. When the ECU 102 determines that the locking mechanism 70 is in the locked position, the ECU 102 may permit operation of the atherectomy device 10 by allowing the current source 19 to send a current to either of the first electromagnetic junction 17 and the second electromagnetic junction 18.

The input 108 may send signals to the ECU 102 indicative of whether to start and stop operation of the atherectomy device 10. When the ECU 102 receives a signal from the input 108 indicative of a desire to start operation of the atherectomy device 10, the ECU 102 may activate the current source 19, thereby sending a current to the first electromagnetic junction 17 and the second electromagnetic junction 18. The current sent to the first electromagnetic junction 17 and the second electromagnetic junction 18 may be an alternating current whose relative phases are selected such that the first electromagnetic junction 17 and the second electromagnetic junction 18 alternate in attracting or repelling the magnetically actuated beads 14. When the ECU 102 receives a signal from the input 108 indicative of a desire to stop operation of the atherectomy device 10, the ECU 102 may deactivate the current source 19, stopping the flow of current to the first electromagnetic junction 17 and the second electromagnetic junction 18.

The input 108 may send a signal to the ECU 102 indicative of a desired amount of current to supply to the electromagnets. When the ECU 102 receives the signal indicative of the desired amount of current, the ECU 102 may change the amount of current output from the current source 19 to the desired amount of current. The desired amount of current may be selected from a set of predetermined amounts associated with predetermined speeds of the magnetically actuated beads 14. The desired amount of current may be individually input via the input 108, where a user may input a specific speed. The input 108 may be a user input device, such as a computer, touch screen, push buttons, etc.

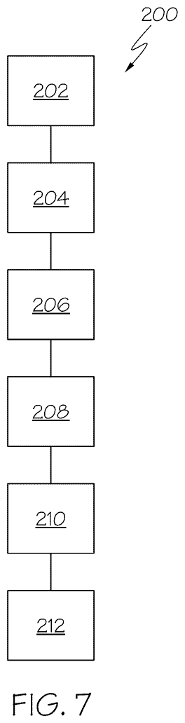

Referring to FIG. 7, a method 200 of operating the atherectomy device 10 is described. One or more steps of the following method may be performed by the ECU 102 of the atherectomy device 10. The atherectomy device 10 may initially be in an OFF state, with the locking mechanism 70 in the unlocked position, and the expansion mechanism 50 in the retracted position 90 such that the atherectomy device 10 may traverse through body vessels with the magnetically actuated beads 14 positioned close to the catheter 12. In the OFF state, the current source 19 does not provide a current to the electromagnets. At step 202, the atherectomy device 10 may be provided into a body vessel 150 of a patient, and advanced through the body vessel 150 to a treatment location. The treatment location may be a part of the body vessel 150 that includes a buildup of calcific plaque. At step 204, the expansion mechanism 50 may be moved within the catheter 12 in the axial direction via the actuator 51 from the retracted position 90 to the partially extended position 92. The movement of the expansion mechanism 50 radially inward of the base 40 of each of the magnetically actuated beads 14 moves one of the first section 52, the second section 56, and the third section 60 between the bases 40 of the magnetically actuated beads 14. The contact between the expansion mechanism 50 and the base 40 of each of the magnetically actuated beads 14 displaces the magnetically actuated beads 14 radially outward to move the magnetically actuated beads 14 from the retracted position 90 to the partially extended position 92. In the partially extended position 92, the magnetically actuated beads 14 extend radially outward from the catheter 12 to contact the plaque in the body vessel 150. At step 206, the locking mechanism 70 may be moved from the unlocked position to the locked position via the locking actuator 71, preventing the expansion mechanism 50 from moving axially, and preventing the magnetically actuated beads 14 from moving from the extended position 94 to the retracted position 90. When the locking mechanism 70 is moved into the locked position, the sensor 106 may send a signal to the ECU 102 indicative of the locking mechanism 70 being in the locked position.

At step 208, the input 108 may send a signal to the ECU 102 indicative of a desire to start operation of the atherectomy device 10. When the ECU 102 receives the signal indicative of a desire to start operation of the atherectomy device 10, the current source 19 may supply an alternating current to the first electromagnetic junction 17 and the second electromagnetic junction 18 to move the magnetically actuated beads 14 along the length of the catheter 12 between the first electromagnetic junction 17 and the second electromagnetic junction 18. As used herein, the term “alternating current” refers to a current that is supplied to only one of the first electromagnetic junction 17 and the second electromagnetic junction 18 at a time so that the magnetically actuated beads 14 are attracted or repelled from one of the first electromagnetic junction 17 and the second electromagnetic junction 18 in the axial direction, and does not correspond to the type of current supplied to the electromagnetic junctions 17, 18 (e.g., AC or DC). In embodiments, the current source may provide current to each of the first electromagnetic junction 17 and the second electromagnetic junction 18 at the same time so long as the magnetically actuated beads 14 continue to move in the axial direction and oscillate between the first electromagnetic junction 17 and the second electromagnetic junction 18. As such, the current source 19 may provide the current to the electromagnetic junctions 17, 18 as either of AC or DC, while still supplying an alternating current to cause the magnetically actuated beads 14 to oscillate between the first electromagnetic junction 17 and the second electromagnetic junction 18. Specifically, the current source 19 may alternate the current sent to the first electromagnetic junction 17 and the second electromagnetic junction 18 by supplying a first current to the first electromagnetic junction 17 from the current source 19 without supplying a current to the second electromagnetic junction 18, then supplying a second current to the second electromagnetic junction 18 from the current source 19 without supplying the first current to the first electromagnetic junction 17. At step 210, the input 108 may send a signal to the ECU 102 indicative of a desired amount of current to supply to the electromagnets. When the ECU 102 receives the signal indicative of a desired amount of current, the current source 19 may change the amount of either of the first current supplied to the first electromagnetic junction 17 and the second current supplied to the second electromagnetic junction 18 to the desired amount.

At step 212, the locking mechanism 70 may be moved from the locked position to the unlocked position via the locking actuator 71. The expansion mechanism 50 may then be moved axially from the partially extended position 92 to either the retracted position 90 or the extended position 94 via the actuator 51 to move the magnetically actuated beads 14 radially either away from or toward the centerline 11 of the catheter 12.

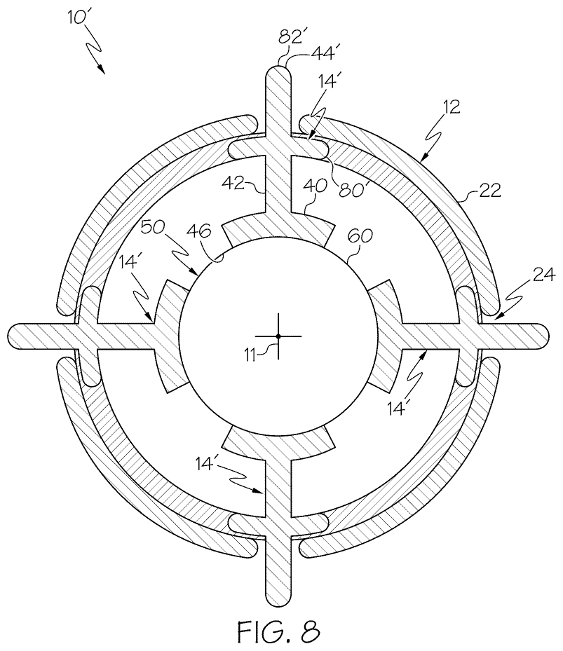

Referring now to FIG. 8, another atherectomy device 10′ is depicted. It should be appreciated that the atherectomy device 10′ is similar to the atherectomy device 10 discussed above. Therefore, like reference numerals will be used to discuss like parts. Accordingly, the atherectomy device 10′ includes the catheter 12, the magnetic drive mechanism 16, the expansion mechanism 50, the locking mechanism 70, and one or more magnetically actuated beads 14′. It should be appreciated that the magnetically actuated beads 14′ differs from the magnetically actuated beads 14 discussed herein such that magnetically actuated beads 14′ each may include a flange 80′ that extends from the shaft 42, a protrusion 82′ extending radially outward from the flange 80′ in the direction of extension of the shaft 42, and an abrasive surface 44′ positioned at the protrusion 82′. The abrasive surface 44′ may extend over the entirety of the protrusion 82′ extending from the flange 80′. The flange 80′ of the magnetically actuated beads 14′ is shaped to be positioned within the lumen 26 of the catheter body 22 with the protrusion 82′ extending out of the catheter body 22 through the openings 24. The flange 80′ may extend around the entire circumference of the shaft 42. The flange 80′ may extend from the shaft 42 to have a width that is greater than a width of the openings 24. The width of the flange 80′ may be greater than the width of the openings 24 to prevent the magnetically actuated beads 14′ from exiting the catheter body 22. The protrusion 82′ may have a width that is lesser than the width of the openings 24 to allow the protrusion 82′ to extend out of the catheter body 22 through the openings 24.

In FIG. 8, the atherectomy device 10′ is depicted with the expansion mechanism 50 in the extended position 94. However, similar to the atherectomy device 10, the expansion mechanism 50 of the atherectomy device 10′ may be movable between the retracted position 90, the partially extended position 92, and the extended position 94. In the extended position 94, the flanges 80′ may contact the catheter body 22 around the openings 24, such that the flanges 80′ restrict the magnetically actuated beads 14′ from exiting the lumen 26 of the catheter body 22. In the retracted position, the magnetically actuated beads 14′ may be completely housed within the catheter 12.

### The Following Clauses Also Relate to the Present Disclosure

An atherectomy device comprising: a catheter having a proximal end and a distal end; at least one magnetically actuated bead movably coupled to the catheter at the distal end, the at least one magnetically actuated bead comprising an abrasive surface for performing an atherectomy procedure; a first electromagnet coupled to the distal end of the catheter at a position proximal to the at least one magnetically actuated bead; and a second electromagnet coupled to the distal end of the catheter at a position distal to the at least one magnetically actuated bead, wherein the first electromagnet and the second electromagnet are configured to move the at least one magnetically actuated bead along a length of the catheter.

The atherectomy device of the preceding clause, wherein: a first current applied to the first electromagnet moves the at least one magnetically actuated bead toward the first electromagnet, and a second current applied to the second electromagnet moves the at least one magnetically actuated bead toward the second electromagnet.

The atherectomy device of any of the preceding clauses, further comprising a current source coupled to the first electromagnet and the second electromagnet and configured to alternate delivery of current between the first electromagnet and the second electromagnet to oscillate the at least one magnetically actuated bead between the first electromagnet and the second electromagnet.

The atherectomy device of any of the preceding clauses, further comprising an electronic control unit communicatively coupled to the current source, the electronic control unit being configured to control an amount of the current from the current source to selectively provide the current from the current source to the first electromagnet and the second electromagnet, wherein controlling the amount of the current controls a speed of the at least one magnetically actuated bead.

The atherectomy device of any of the preceding clauses, further comprising: an expansion mechanism movably coupled to the catheter, and configured to move in a longitudinal direction along the length of the catheter between a retracted position and an extended position, wherein moving the expansion mechanism to the retracted position radially displaces the at least one magnetically actuated bead from a centerline of the catheter.

The atherectomy device of any of the preceding clauses, wherein: the catheter comprises a catheter body defining a lumen and an opening extending through a sidewall of the catheter body to the lumen; the expansion mechanism is positioned within the lumen, in the retracted position, the at least one magnetically actuated bead is at least partially positioned within the lumen, and in the extended position, the at least one magnetically actuated bead extends out of the opening in the catheter, and is spaced apart from an outer surface of the catheter body.

The atherectomy device of any of the preceding clauses, wherein the expansion mechanism comprises an elongated member having a tapered surface in a distal to proximal direction slidably engaged with the at least one magnetically actuated bead.

The atherectomy device of any of the preceding clauses, wherein the at least one magnetically actuated bead comprises one of a magnetic and a ferromagnetic material.

An atherectomy device comprising: a catheter having a proximal end and a distal end; at least one magnetically actuated bead movably coupled to the catheter at the distal end, the at least one magnetically actuated bead comprising a shaft, a base having a contact surface, and an abrasive surface for performing an atherectomy procedure, the abrasive surface is coupled to an end of the shaft opposite the base; an expansion mechanism coupled to the catheter, the expansion mechanism comprises a first section having a first thickness, and a second section having a second thickness greater than the first thickness, the expansion mechanism configured to move between a retracted position and an extended position, in the retracted position, the contact surface of the at least one magnetically actuated bead contacts the first section of the expansion mechanism, in the extended position, the contact surface of the at least one magnetically actuated bead contacts the second section of the expansion mechanism, and moving the expansion mechanism from the retracted position toward the extended position moves the at least one magnetically actuated bead away from a centerline of the catheter.

The atherectomy device of the preceding clause, further comprising at least one electromagnet coupled to the distal end of the catheter, the at least one electromagnet configured to move the at least one magnetically actuated bead along a length of the catheter, wherein the at least one electromagnet comprises: a first electromagnet coupled to the distal end of the catheter at a position proximal the at least one magnetically actuated bead; and a second electromagnet coupled to the distal end of the catheter at a position distal to the at least one magnetically actuated bead.

The atherectomy device of any of the preceding clauses, wherein: a first current applied to the first electromagnet moves the at least one magnetically actuated bead toward the first electromagnet, and a second current applied to the second electromagnet moves the at least one magnetically actuated bead toward the second electromagnet.

The atherectomy device of any of the preceding clauses, further comprising a current source coupled to the first electromagnet and the second electromagnet and configured to alternate delivery of current between the first electromagnet and the second electromagnet to oscillate the at least one magnetically actuated bead between the first electromagnet and the second electromagnet.

The atherectomy device of any of the preceding clauses, further comprising an electronic control unit communicatively coupled to the current source, the electronic control unit being configured to control an amount of the current from the current source to selectively provide the current from the current source to the first electromagnet and the second electromagnet, wherein controlling the amount of the current controls a speed of the at least one magnetically actuated bead.

The atherectomy device of any of the preceding clauses, wherein the at least one magnetically actuated bead comprises one of a magnetic and a ferromagnetic material.

The atherectomy device of any of the preceding clauses, wherein: the catheter comprises a catheter body defining a lumen and an opening extending through a sidewall of the catheter body to the lumen; the expansion mechanism is positioned within the lumen, in the retracted position, the base of the at least one magnetically actuated bead is at least partially positioned within the lumen, and in the extended position, the at least one magnetically actuated bead extends out of the opening in the catheter, and is spaced apart from an outer surface of the catheter body.

The atherectomy device of any of the preceding clauses, wherein the expansion mechanism comprises an elongated member having a tapered surface in a distal to proximal direction slidably engaged with the at least one magnetically actuated bead.

The atherectomy device of any of the preceding clauses, wherein the at least one magnetically actuated bead comprises a pair of magnetically actuated beads positioned radially diametrically opposite one another across the centerline of the catheter.

A method of operating an atherectomy device, the method comprising: providing the atherectomy device, the atherectomy device comprising: a catheter having a proximal end and a distal end; at least one magnetically actuated bead movably coupled to the catheter at the distal end, the at least one magnetically actuated bead comprising an abrasive surface for performing an atherectomy procedure; a first electromagnet coupled to the distal end of the catheter at a position proximate the at least one magnetically actuated bead; and a second electromagnet spaced coupled to the distal end of the catheter at a position distal to the at least one magnetically actuated bead, wherein the first electromagnet and the second electromagnet are configured to move the at least one magnetically actuated bead along a length of the catheter; and supplying a current to the first electromagnet and the second electromagnet to move the at least one magnetically actuated bead along the length of the catheter.

The method of the preceding clause, further comprising: supplying a first current to the first electromagnet from a current source; supplying a second current to the second electromagnet from the current source; and changing an amount of either of the first current supplied to the first electromagnet and the second current supplied to the second electromagnet.

The method of either of the preceding clauses, further comprising: adjusting an expansion mechanism slidably positioned within the catheter to move the at least one magnetically actuated bead in a direction away from or toward a centerline of the catheter.

It should now be understood that embodiments of the present disclosure are directed to devices, systems, and methods for an atherectomy device including a catheter, at least one magnetically actuated bead including an abrasive element for abrading plaque, electromagnets for oscillating the bead along a length of the catheter, and an expansion mechanism for adjusting the radial position of the magnetically actuated bead.

It is noted that the term “substantially” may be utilized herein to represent the inherent degree of uncertainty that may be attributed to any quantitative comparison, value, measurement, or other representation. This term is also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. An atherectomy device comprising:
a catheter having a proximal end and a distal end;
at least one magnetically actuated bead movably coupled to the catheter at the distal end, the at least one magnetically actuated bead comprising an abrasive surface for performing an atherectomy procedure;
a first electromagnet coupled to the distal end of the catheter at a position proximal to the at least one magnetically actuated bead; and
a second electromagnet coupled to the distal end of the catheter at a position distal to the at least one magnetically actuated bead, wherein the first electromagnet and the second electromagnet are configured to move the at least one magnetically actuated bead along a length of the catheter.

2. The atherectomy device of claim 1, wherein:
a first current applied to the first electromagnet moves the at least one magnetically actuated bead toward the first electromagnet, and
a second current applied to the second electromagnet moves the at least one magnetically actuated bead toward the second electromagnet.

3. The atherectomy device of claim 2, further comprising a current source coupled to the first electromagnet and the second electromagnet and configured to alternate delivery of current between the first electromagnet and the second electromagnet to oscillate the at least one magnetically actuated bead between the first electromagnet and the second electromagnet.

4. The atherectomy device of claim 3, further comprising an electronic control unit communicatively coupled to the current source, the electronic control unit being configured to control an amount of the current from the current source to selectively provide the current from the current source to the first electromagnet and the second electromagnet, wherein controlling the amount of the current controls a speed of the at least one magnetically actuated bead.

5. The atherectomy device of claim 1, further comprising:
an expansion mechanism movably coupled to the catheter and configured to move in a longitudinal direction along the length of the catheter between a retracted position and an extended position, wherein moving the expansion mechanism to the retracted position radially displaces the at least one magnetically actuated bead from a centerline of the catheter.

6. The atherectomy device of claim 5, wherein:
the catheter comprises a catheter body defining a lumen and an opening extending through a sidewall of the catheter body to the lumen;
the expansion mechanism is positioned within the lumen,
in the retracted position, the at least one magnetically actuated bead is at least partially positioned within the lumen, and
in the extended position, the at least one magnetically actuated bead extends out of the opening in the catheter, and is spaced apart from an outer surface of the catheter body.

7. The atherectomy device of claim 6, wherein the expansion mechanism comprises an elongated member having a tapered surface in a distal to proximal direction slidably engaged with the at least one magnetically actuated bead.

8. The atherectomy device of claim 1, wherein the at least one magnetically actuated bead comprises one of a magnetic and a ferromagnetic material.

9. An atherectomy device comprising:
a catheter having a proximal end and a distal end;
at least one magnetically actuated bead movably coupled to the catheter at the distal end, the at least one magnetically actuated bead comprising a shaft, a base having a contact surface, and an abrasive surface for performing an atherectomy procedure, the abrasive surface is coupled to an end of the shaft opposite the base;
an expansion mechanism coupled to the catheter, the expansion mechanism comprises a first section having a first thickness, and a second section having a second thickness greater than the first thickness, the expansion mechanism configured to move between a retracted position and an extended position,
in the retracted position, the contact surface of the at least one magnetically actuated bead contacts the first section of the expansion mechanism,
in the extended position, the contact surface of the at least one magnetically actuated bead contacts the second section of the expansion mechanism, and
moving the expansion mechanism from the retracted position toward the extended position moves the at least one magnetically actuated bead away from a centerline of the catheter.

10. The atherectomy device of claim 9, further comprising at least one electromagnet coupled to the distal end of the catheter, the at least one electromagnet configured to move the at least one magnetically actuated bead along a length of the catheter, wherein the at least one electromagnet comprises:
a first electromagnet coupled to the distal end of the catheter at a position proximal the at least one magnetically actuated bead; and
a second electromagnet coupled to the distal end of the catheter at a position distal to the at least one magnetically actuated bead.

11. The atherectomy device of claim 10, wherein:
a first current applied to the first electromagnet moves the at least one magnetically actuated bead toward the first electromagnet, and
a second current applied to the second electromagnet moves the at least one magnetically actuated bead toward the second electromagnet.

12. The atherectomy device of claim 11, further comprising a current source coupled to the first electromagnet and the second electromagnet and configured to alternate delivery of current between the first electromagnet and the second electromagnet to oscillate the at least one magnetically actuated bead between the first electromagnet and the second electromagnet.

13. The atherectomy device of claim 12, further comprising an electronic control unit communicatively coupled to the current source, the electronic control unit being configured to control an amount of the current from the current source to selectively provide the current from the current source to the first electromagnet and the second electromagnet, wherein controlling the amount of the current controls a speed of the at least one magnetically actuated bead.

14. The atherectomy device of claim 9, wherein the at least one magnetically actuated bead comprises one of a magnetic and a ferromagnetic material.

15. The atherectomy device of claim 9, wherein:
the catheter comprises a catheter body defining a lumen and an opening extending through a sidewall of the catheter body to the lumen;
the expansion mechanism is positioned within the lumen,
in the retracted position, the base of the at least one magnetically actuated bead is at least partially positioned within the lumen, and
in the extended position, the at least one magnetically actuated bead extends out of the opening in the catheter, and is spaced apart from an outer surface of the catheter body.

16. The atherectomy device of claim 9, wherein the expansion mechanism comprises an elongated member having a tapered surface in a distal to proximal direction slidably engaged with the at least one magnetically actuated bead.

17. The atherectomy device of claim 9, wherein the at least one magnetically actuated bead comprises a pair of magnetically actuated beads positioned radially diametrically opposite one another across the centerline of the catheter.

18. A method of operating an atherectomy device, the method comprising:
providing the atherectomy device, the atherectomy device comprising:
a catheter having a proximal end and a distal end;
at least one magnetically actuated bead movably coupled to the catheter at the distal end, the at least one magnetically actuated bead comprising an abrasive surface for performing an atherectomy procedure;
a first electromagnet coupled to the distal end of the catheter at a position proximate the at least one magnetically actuated bead; and
a second electromagnet coupled to the distal end of the catheter at a position distal to the at least one magnetically actuated bead, wherein the first electromagnet and the second electromagnet are configured to move the at least one magnetically actuated bead along a length of the catheter; and
supplying a current to the first electromagnet and the second electromagnet to move the at least one magnetically actuated bead along the length of the catheter.

19. The method of claim 18, further comprising:
supplying a first current to the first electromagnet from a current source;
supplying a second current to the second electromagnet from the current source; and
changing an amount of either of the first current supplied to the first electromagnet and the second current supplied to the second electromagnet.

20. The method of claim 18, further comprising:
adjusting an expansion mechanism slidably positioned within the catheter to move the at least one magnetically actuated bead in a direction away from or toward a centerline of the catheter.
